(19) United States
(12) Patent Application Publication
(10) Pub. No.: US 2012/0234332 A1
(43) Pub. Date: Sep. 20, 2012

(54) SNORING AND OBSTRUCTIVE SLEEP APNEA PREVENTION AND TREATMENT DEVICE

(76) Inventor: Totada R. Shantha, Stone Mountain, GA (US)

(21) Appl. No.: 13/485,955

(22) Filed: Jun. 1, 2012

(51) Int. Cl. A61F 5/56; A61M 16/00

(52) U.S. Cl. 128/848

(57) ABSTRACT

A anti-snoring and anti-obstructive sleep apnea apparatus has a plate or metal frame tongue shelf splint to prevent the flaccid tongue falling back, a palate shelf splint projection to elevate the soft palate and prevents its vibration, incisors teeth receptacles or pockets sockets for jaw displacer to hold the mandible moved forwards, and to prevent it falling back held between the bite block, catheter-tubing to administer oxygen supplementation from the external source which reduces air turbulence and promotes nasal breathing, a submental suprahyoid muscle stimulator placed below the tongue, and above the mucous membrane. An injection port is provided to administer any therapeutic agents and local anesthetics to reduce the sensitivity of the tongue and oral cavity mucus membrane lining to the foreign objects. This device is used to deliver therapeutic agents to prevent and to treat halitosis and various diseases.

### FIELD OF THE INVENTION

The present invention relates to an apparatus to be used for therapy for patients suffering from snoring and obstructive sleep apnea (OSA) syndromes. These conditions are due to a breathing malfunction occurring during sleep which results in narrowing and/or obstruction of the upper respiratory passages. During the therapy, the palatine and tongue muscles are splinted, supported, and to a large extent immobilized to prevent the palate from vibrating and where there is the propensity of the tongue to fall back in the mouth which the therapy prevents during sleep. Such an apparatus may be used to treat halitosis and disease afflictions of the tongue and palate. The current invention relates to an anti snoring and anti obstructive sleep apnea (OSA) devices and their methods of use.

### BACKGROUND OF THE INVENTION

The tongue and soft palate play a major role in production of snoring and obstructive sleep apnea. Their participation causes these symptoms to exist. Hence, it is imperative to have knowledge about these structures which play a major important role in snoring and obstructive sleep apnea.

The word tongue derives from the Old English tunge, which comes from Proto-Germanic tungon. It is able to be used as a metonym, i.e. figure of speech for language, as in the phrase mother tongue. Many languages have the same word for “tongue” and “language”. The tongue is a muscular- neuro-vascular body on the floors of the mouths of most vertebrates. It maneuvers food for mastication which is the primary organ of taste, through papillae and taste buds that cover the surface of the tongue. It is kept moist by saliva, and mucous glands of the mouth which are richly supplied with nerves and blood vessels. In humans a secondary function of the tongue is phonetic articulation. The tongue serves as a natural means of cleaning one's teeth by its all directional movement capacity. In mammals (dogs, cats and other animals), the tongue has a rough surface which is used to clean the fur and body. A dog's tongue acts as a heat regulator.

The average length of the human tongue in adults from the oropharynx to the tip is 10 cm (4 inches). It has 5 intrinsic muscles (Superior, inferior, longitudinal, transverse & vertical) within the tongue that are not attached to any bone where the 4 extrinsic muscles are attached to the bones below the tongue. They are the genio, hyo, stylo, palato-glossus muscles. The primary blood supply to tongue is from the lingual artery, a branch of the external carotid artery. There is a secondary blood supply to the tongue from the tonsillar branch of the facial artery and the ascending pharyngeal artery.

The underneath of the front of the tongue is called the sublingual region where the oral mucosa is very thin with a rich plexus of veins. This sublingual region is used as route of administration of many therapeutic agents. This is the distinct expedient and efficacious route of administration of nitroglycerin to a patient suffering chest pain from angina pectoris. If the tablet is swallowed, the medication is completely neutralized by the detoxification process from the digestive juices and by the liver.

Tongue is one of the most active skeletal muscles mass in the body which is used from the minute one wakes up and then goes to sleep. Hence, the tongue is constantly subjected to trauma intended or unintended. The tongue is constantly moving while eating, drinking and talking as well as during other facial activities. Hence, the tongue is subject to trauma by the teeth and the physical forces of cold and hot temperature of substances as well as exposure to different chemicals consumed like alcohol, acids, alkalis and tooth paste, etc. play a role in affecting the tongue and palate.

The tongue is supplied by many nerves which are sensitive and can cause the gag reflex from a foreign object. Taste sensation for the anterior ⅔ of the tongue is supplied by the Facial nerve (Chorda tympani, CN VII). General sensation of the anterior ⅔ is supplied by the Lingual nerve which is a branch of V3 of the Trigeminal nerve CN V. Taste as well as general sensation for the posterior ⅓ is supplied by the Glossopharyngeal nerve (CN IX). All intrinsic and extrinsic muscles of the tongue are supplied by the Hypoglossal nerve (CN XII), except for one of the extrinsic muscle, palatoglossus that is innervated by CN X of the pharyngeal plexus. The most posterior part of the tongue is supplied by the internal laryngeal nerve, a branch of the vagus nerve. The tongue plays a primary role in the production of obstructive sleep apnea.

The palate forms the roof of the mouth and is made up of two regions-the hard palate in front, the soft palate behind. The hard palate is formed by the palatine processes of the maxillae and the horizontal plates of the palatine bones; behind, it is continuous with the soft palate. The upper surface of the hard palate forms part of the floor of the nasal cavity which is lined by ciliated epithelium,

There are five pairs of palatine muscles of the soft palate that are involved in the movement of the palate and uvula which can participate in production of snoring. They are: 1. Tensor palati; 2. levator palati and 3. Palatopharyngeus from the upper surface, 4. With the uvular muscles within the upper surface. 5. Palatoglossus from the lower surface. The flexible skeleton for the soft palate is provided by the aponeurosis of Tensor palati muscle.

It is the soft palate along with the tongue and oro-laryngo-pharynx that plays a role in snoring and obstructive sleep apnea. The soft palate is suspended from the posterior border of the hard palate, extends downwards, and backwards between the oral and nasal parts of the pharynx. The soft palate consists of mucous membrane enclosing an aponeurosis, muscular fibers, vessels, nerves, lymphoid tissue and mucous glands. Its superior border is attached to the posterior margin of the hard palate, and its sides are blended with the pharynx. Its inferior border is free that contributes to snoring. The uvula is a small conical process, hangs from the middle of its lower border where the two curved folds of mucous membrane, containing muscular fibers (palatoglossal arch), extends laterally and downwards from each side of the base of the uvula. A thin, firm, fibrous lamella, termed the palatine aponeurosis, which supports the muscles and gives strength to the soft palate, is attached to the posterior border of the hard palate and to the inferior surface of the hard palate behind the palatine crest. The muscles of the palate include a levator and a tensor of the palate. The muscles are underlying in the palatoglossal and palatopharyngeal folds which extends into the palate itself and the muscle of the uvula with the exception of the tensor veli palatine. The flexible skeleton for the soft palate is provided by the aponeurosis of Tensor palati muscle. The muscles are innervated by the mandibular nerve of the soft palate that are supplied by nerve fibers which leave the medulla in the cranial part of the accessory nerve which reach the pharyngeal plexus via the vagus nerve.

Snoring, hypopnea and obstructive sleep apnea (OSA) are caused by the vibrating soft palate; soft tissue of the nasal and oral pharynx, relaxed tongue moving backwards towards the oral and laryngpharynx which block the air passageway through the pharynx, or lingual compartment during sleep obstructing air passage through the naso, oro and laryngpharynx. Other causes ncludes; the loose tissue within the mouth cavity including the flaccid tongue, the pharyngeal folding, tonsillar pillars, and the muscular uvula with the soft palate-called the pharyngeal arch that has a propensity to vibrate as tidal air flows past narrow air passages during sleep causing snoring and obstructive sleep apnea.

Snoring is an inspiratory sound arises in the course of person's sleep which is due to the narrowing of the naso, oro and laryngo-pharyngeal airway with inspiratory air flow in the narrow passages. The sounds of snoring are generated by vibration of soft tissues of oropharynx such as the soft palate, uvula, tongue, lips, the posterior faucial pillars of the tonsils, pharyngeal folds, posterior, and lateral pharyngeal wall and epiglottis in the upper airway; soft palate and uvula are the main culprits (FIGS. 1, 2, 3).

Many causes for the narrowing of the nasal pharyngeal airway during sleep exist besides the flaccid soft palate and the tongue role. People who snore rarely make snoring sounds when breathing while awake in the same position that is associated with snoring when asleep (FIGS. 1, 2). The reason being that the wide awake conscious person has watchful control of various muscles of the upper airway to prevent the vibrations that cause snoring to occur (FIG. 1). During sleep, the motor neurons that control skeletal muscles are inhibited from sending instructions to make them active which increase the tone of these muscles. This physiological process in sleep results in flaccid muscles that permit soft tissue to sag and collapse into the pharyngeal wall that results in snoring with OSA strikes (FIGS. 2, 3).

It has been estimated that up to 45% of all adults snore sporadically with about 25% being constant snorers. It is known that the snoring increases with advancing age, This has been observed that about 50% of men and 40% of women are habitual snorers by the age of 60 (Lugaresi et al, “Snoring: Pathogenic, Clinical and Therapeutic Aspects”, Reported in Principles and Practice of Sleep Medicine (Kryger et al, Editors 1989) at pp. 494-500).

One needs to discriminate the difference between non obstructive snorers (FIG. 2) from the obstructive sleep apnea snorers ((FIG. 3) and hypopnea (FIG. 2). Hypopnea is a medical term that involves episodes of shallow breathing or an abnormally low respiratory rate. This may not be due to naso-oro-laryngo-pharyngeal airways. This differs from sleep apnea in that there remains some flow of air. Hypopnea events may happen while asleep or while awake. It's abnormally shallow breathing lasting at least ten seconds. In the context of diagnosis and treatment of sleep disorders, a hypopnea event is not considered to be clinically significant unless there is a 30% or greater reduction in air flow lasting for 10 seconds or longer with an associated 4% or greater desaturation in the person's oxygen levels, or it results in arousal or fragmentation of sleep. During hypopnea, there is airflow, through a much reduced level, which leads to not getting enough oxygen. The apnea-hypopnea index or respiratory disturbance index (AHI) is an index of severity that combines apneas and hypopneas. Combining them both gives an overall severity of sleep apnea including sleep disruptions and oxygen desaturation (a low level of oxygen in the blood).

An apnea index or Al shows the average number of apneas per hour of sleep. A hypopnea index or HI shows the average number of hypopneas per hour of sleep. An apnea and hypopnea index or AHI shows the average number of apneas and hypopnea per hour of sleep. Some doctors use the term of respiratory disturbance index or RDI, instead of AHI. The apnea-hypopnea index, like the apnea index and hypopnea index, is calculated by dividing the number of apneas and hypopneas by the number of hours of sleep. AHI values are categorized as 5-15 Mild, 15-30 Moderate, and above 30 listed as Severe. Example: Apnea+Hypopnea divided by actual sleep time, and then multiply by 60. 200 Apnea 200 Hypopnea=400 Total Events; 420 Actual Sleep time (7 hours). Divide 400 by 420=0.95×60(minutes per hour)=57 AHI (Severe OSA).

The physiological terms used to describe various types of breathing are associated with snoring with or without obstructive sleep apnea breathing difficulties are: Eupnea-normal breathing, Apnea—absence of breathing, Bradypnea-decreased breathing rate, Dyspnea or shortness of breath-sensation of respiratory distress, Hyperaeration/Hyperinflation-increased lung volume, Hyperpnea-fast and deep breathing, Hyperventilation—increased breathing that causes CO₂ loss, Hypopnea-slow and shallow breathing, Hypoventilation-decreased breathing that causes CO₂ gain, and Labored breathing-physical presentation of respiratory distress. Obstructive sleep apnea (OSA) is due to complete blockage of air to larynx due to mechanical soft tissue blockage (FIG. 3) by Naso-Oro-laryngo-pharyngeal tissue.

TYPES OF SLEEP APNEA: There are three types of sleep apnea. They are as follows:

1. Obstructive sleep apnea (OSA) is the common form of the condition when the tissues of the naso-oro-laryngeal-pharynx obstruct breathing during sleep. These pauses in breathing are called apneas (literally, “without breath”), usually last 20 to 40 seconds. There are more than 20 million suffer from OSA in US and its occurrence in adult population is estimated to be 3-4% in women and 6-7% in males. People who gain weight, develop obesity, craniofacial syndromes (mostly genetic), repair of cleft palpate, Down syndrome, small mandible etc. have a higher risk of developing obstructive sleep apnea than most individuals. Our invention is mainly intended to treat these conditions causing obstructive sleep apnea.

2. Central sleep apnea is due to a neurological condition as a result of a head injury, stroke, or various central nervous system disorder, and heart failure. Patients with central sleep apnea should avoid using sedatives, narcotics, and alcohol. Treating the primary etiology will in most cases eliminates the condition. Unfortunately the primary etiology may be terminal.

3. Mixed sleep apnea is due to physical oropharyngeal airflow obstruction associated with central (CNS) etiology. It is a rare condition that is the most dangerous form of sleep apnea. Therefore, it is the most difficult to treat. The present invention is provides a treatment for this form of obstructive sleep apnea.

Symptoms of Obstructive Sleep Apnea are: Frequent cessation of breathing (apnea) during sleep. A sleeping spouse or companion may notice repeated silences from your side of the bed with sudden awakenings to restart breathing with choking or gasping during sleep to get air. Loud snoring and awakening in a sweat during the night due to lack of oxygen with an increase of carbon dioxide build up, waking up restless in the morning after a night's sleep with or without headaches, sore throat, or dry mouth in the mornings, daytime sleepiness including falling asleep at improper times, when driving to work, at meeting and conferences with fatigue, mood changes like irritability, anxiety, depression, trouble concentrating, forgetfulness reduced and dwindling sex drive, unexplained weight gain; increased urination and/or nocturia, frequent heartburn, gastro-esophageal reflux disease (GERD), and heavy night sweats can strike.

Studies by Lee et al. has shown that the oxygen desaturation sleep events were detected in all patients with OSA, but not in simple snorers (Lee C H, Moil-I, Kim B J, Kong I G, Yoon I Y, Chung S, Kim J W, Kim J W, Arch Otolaryngol Head Neck Surg. Evaluation of soft palate changes using sleep video fluoroscopy in patients with obstructive sleep apnea. 2009 eb;135(2):168-72). When awake, inspiratory efforts increased the length and angle of the soft palate (SP) in patients with OSA but not in simple snorers. Elongation and angulations were greatest during desaturation sleep events and least during awake (FIGS. 1, 3). In normal oxygenation events, changes in the soft palate (SP) were significantly larger in patients with OSA than in simple snorers (P <0.01 for SP length; P=0.03 for SP angle). These studies showed that the SP was considerably elongated and angulated in patients with OSA even when awake. Hence the treatment of snoring should be differentiated for the sake of treatment: 1. to prevent production of sound during sleep, 2. the obstructive sleep apnea with serious health consequences. Our invention is intended treatments of the both conditions.

There are no effective FDA approved drug treatments for obstructive sleep apnea. Nevertheless, a clinical trial of anti depressants mirtazapine (Brand name: Remeron, Avanza, Zispin) has shown promising results in the treatment of Obstructive Sleep Apnea, but it causes weight gains and sedation (“First Effective Drug for Sleep Disorder Identified”. ScienceDaily.com.June 2003). It is a tetra cyclic antidepressant (TeCA) used primarily in the treatment of depression. The drug may treat as a hypnotic, antiemetic, appetite stimulant, and for the treatment of anxiety. Mirtazapine is not a SSRI reuptake inhibitor. It disinhibits dopamine and norepinephrine activity in various parts of the brain in the pleasure centers such as the ventral tegmental area (VTA) which causes a pronounced antidepressant and anxiolytics response due to release of the neurotransmitters dopamine and norepinephrine. Beside its close analogues, mianserin and setiptiline, mirtazapine is one of the small number of noradrenergic and specific serotonergic antidepressants (NaSSAs) that can be tired on OSA.

Serotonin uptake inhibitors (SSRI) such as fluoxetine, tryptophan, protriptyline, oral methylxanthine, and theophylline (chemically similar to caffeine), amphetamines stimulants; to anti-narcoleptic medications such as modafinil are tried. A course of anti-inflammatory steroids such as prednisone (or another glucocorticoid drug) is given to reduce the lymphoid tissue of the naso-oropharyngeal air passages if enlargement of the lymphoid tissue is found when the allergic conditions are suspected.

A basic treatment for snoring and obstructive sleep apnea involves having the patient sleep in the prone position or on his/her side. This is stimulated by sewing an object into the back of the snorer's clothes. In an obese person treatment includes weight loss. These treatments there are recommendation that the patient avoid use of CNS depressing drugs, cigarettes, or alcohol prior to bedtime which prevents or reduces the loss of oropharyngeal muscle tone.

Obstruction due to enlarged tonsils or adenoids may need to be removed. In some cases surgical repair of a deviated nasal septum has been shown to improve snoring. Snoring can be due to genetics with some being predisposed towards an anatomical narrowing across the nasal-oral-laryngeal-pharynx. A reduced pharyngeal passageway may be caused by a lack of muscle tone. Other anatomical conditions contributing to the narrowing of the naso-oro-laryngo-pharyngeal passageway includes choanal atresia, chrono polyp, nasal septal deviation, nasal and pharyngeal cysts, macroglossia, retrognathia, micrognathia and countless other etiologies. (Leung et al, “The ABZzzz's of Snoring”, Post Graduate Medicine; Sep. 1, 1992).

Snoring and OSA might be aggravated by alcohol drinks or drugs (such as tranquilizers, hypnotic, sleeping pills, and antihistamines) taken prior to bedtime. Smoking is held responsible for snoring. The cigarettes may irritate the mucus membranes of the upper airway and oropharynx which causes swelling and increased mucus production. When snoring is caused by nasal allergy or an upper respiratory tract infection, these conditions may be treated with antiallergenic treatment (Douglas N.J. “The Sleep Apnoea/Hypopnoea Syndrome And Snoring”, British Medical journal, 1993, Vol. 306:1057-60; Leung et al, “The ABZzzz's of Snoring” Post Graduate Medicine (Sep. 1, 1992).

Anti-snoring and anti OSA devices abound in the medical device market. Some of them are shown to be effective when they pull or hold the mandible (lower jaw) forward and upward and elevate the tongue when the muscles of the mandible relax, that the tongue does not occlude the air passageway drifting inferiorly and posteriorly while sleeping to prevent the passage of air (FIG. 1). Most anti-snoring devices accomplish this task by moving the lower jaw forward and holding that position against a rigid upper dental component which is fixed to the upper teeth in the immobile maxilla and to the lower teeth in the mandible. The disadvantages in using the above prior art devices are that they require expert qualified licensed lab services for fitting of the anti-snoring device to the user's mouth. Such devices could cause permanent irremediable changes in the bite of the user and permanently alter the jaw position. This requires a dentist to closely monitor anti snoring device fitting. There is a need for an anti-snoring device that does not rigidly bind to the dental structures of the user's mouth that does not require professional supervision or assistance in its fabrication, monitoring of the dental bite changes, and mandibular changes. In addition, the anti-snoring device should not pit the lower jaw against the upper jaw. These devices do not include an intra oral dental overlay to support the tongue against the palate and keep the palate of the user's mouth from reverberating (snoring) during mouth breathing. Our inventions overcomes these draw backs.

Snoring and OSA can be managed by the use of a positive pressure generator and facemask. In this procedure, a mask that covers nose and mouth or nose or mouth and delivers air under pressure. The standard method is known as “Continuous Positive Airway Pressure” (CPAP) treatment that requires the patient to wear a mask which air is blown into the nostrils to keep the airway open. Patient compliance is poor due to discomfort and side effects. These machines pump air through a hose and nose/mouth face mask to keep air passages clear and open. CPAP pneumatically splints the upper airway. Use of the devices, can cause the subject to become non complaint due to difficulty in its use due to discomfort problems during sleep. Problems may occur with CPAP include: restless sleep, dryness of nose, throat, and nasopharyngeal tract, cough, excessive dreaming during early use, nasal congestion, runny nose, sneezing, irritation of the eyes and the skin on the face, abdominal bloating, and leaks around the mask because when it does not fit properly.

The person may be able to limit or stop some of the side effects. The doctor may be able to adjust your CPAP to reduce or eliminate problems which are to make sure the mask or nasal prongs fit you properly where air should not leak around the mask. The use of a humidifier or a corticosteroid nasal spray medicine to reduce nasal congestion, irritation, and drainage.

User of this method of treatment may need to talk to a doctor about trying a CPAP machine that will help to reduce discomfort caused by too much constant pressure in the user's nose. If this does not improve discomfort, ask your physician about trying a bi-level positive airway pressure machine (BiPAP-VPAP or variable positive airway pressure), which uses a different air pressure when you breathe in when you exhale. BiPAP may work better than standard CPAP for treating obstructive sleep apnea in people who have heart failure. BiPAP machines are more expensive than CPAP machines.

When one is using CPAP or BiPAP, the person needs to see their doctor and sleep specialist regularly. There may be a need for more sleep studies to adjust the CPAP machine and check, whether, the treatment is working. The sleep studies and the CPAP machines are expensive. A patient can rent a CPAP machine before purchasing one. The most common problem with CPAP is lack of compliance. This means that people do not use the machine every night because the machine is uncomfortable. The patient may remove the machine as they sleep which leaves the patient sleepy the next day due to repeated interruption during sleep.

A more recent treatment option to obstructive sleep apnea includes the implantation of rigid inserts in the soft palate to provide structural support, is both invasive which is only effective for mild to moderate cases of obstructive sleep apnea. Alternative treatments are even more invasive and drastic: including tracheostomy, genioglossus advancement or stimulator, hyoid suspension, tongue reposition, and tissue ablation (somnoplasty or uvulopalatopharyngoplasty (UPPP)).

If all else fails, sleep apnea can be effectively treated by maxillomandibular advancement. It is a complex operation in which the maxilla holding the upper teeth and the mandible holding the lower teeth are surgically cut and moved so that the lower part of your face is moved forward approximately 12 millimeters. In this complex surgical procedure, the airway in the back of the throat is expanded to relieve the obstructive sleep apnea. This undertaking is advised only for disabling obstructive sleep apnea patients in whom other treatments have failed. Reduction of the size of the soft palate, laser-assisted uvulopalatoplasty, reduction of the tongue base either with laser excision or radiofrequency ablation, Genioglossus Advancement, Hyoid Suspension in which the hyoid bone in the neck are attempted to treat this condition. In rare intractable cases, tracheostomy is the only effective treatment for sleep apnea.

Due to many associated disadvantages, complications and high failure rate, these tissue ablation methods and radical surgeries need to be considered as a last resort. Other options for treating snoring are found with surgical techniques where there are removal of enlarged adenoids, tonsils, and host of other therapies are recommended. Surgical removal of the uvula, distal portion of the soft palate, the anterior tonsillar pillars, and the redundant lateral pharyngeal wall mucosa are said to increase the size of the air passageway allowing unobstructed movement of air through the pharynx. Rates of success of the uvulopalatopharyngoplasty are reported to be in a range from 15% to 65%. (Douglas, “The Sleep Apnoea/Hypopnoea Syndrome And Snoring”, British Medical journal, 1993, Vol. 306:1057-60). In some instances, surgical repair of a deviated nasal septum has been shown to improve snoring but not OSA.

Snoring and obstructive sleep apnea results in exhaustion that results from lack of sleep and interfering at work, and while driving is a problem. Obstructive sleep apnea causes high blood pressure, depression, irregular heartbeats , heart failure, coronary artery disease, and stroke. If the person is overweight, bariatric surgery may help to lose weight which may improve sleep apnea.

Snoring and obstructive sleep apnea patients with decreased pulmonary function such as emphysema, asthma, chronic obstructive lung diseases (COPD), and congestive heart failure have been shown to suffer from severe apnea. Cessation of breathing during snoring, or obstructive sleep apnea results in lack of oxygen due to an obstructed nasopharyngeal passageway that deprives the body of sufficient oxygen which the oxygen de saturation arises. Lack of oxygen may cause the brain to awaken the sleeper to take a breath without fully awaking. This may happen dozens and even hundreds of times a night. The snorer and OSA patients do not get sufficient sleep. When being aroused from deep REM sleep on a repetitive basis increases heart rate and blood pressure with associated increases the risk of heart attack and stroke. Furthermore, due to narcolepsy resulting from exhaustion can cause a lack of attention for the snorer and OSA sufferers during waking hour's causing drop in productivity and accident proneness at work, driving and other daily activities.

U.S. Pat. No. 5,569,679 discloses the use of nasal solution 10%-16% of methylsulfonylmethane (MSM) drops for the treatment of anti snoring method. It is a nasal spray, too simplistic to a complicated anatomically related snoring with or without obstructive sleep apnea whose pathophysiology is not in the nose.

U.S. Pat. No. 5,921,241 discloses an anti-snoring device including a moldable dental overlay for covering the lower teeth of the user and for maintaining the tongue in contact with the palate to prevent air flow from causing the palate to reverberate during mouth breathing.

U.S. patent application Publication Number: US 2004/0153127 A1 invention provides electrical stimulation that causes the oropharyngeal muscles to contract during sleep using one or more micro stimulators injected into or near these muscles or the nerves which innervate them.

U.S. patent application Publication Number: US 2007/0233276 A1 describes the method and apparatus include placing a tissue contractor within the tongue tissue. This is invasive procedure and may create discomfort and complication after surgery.

U.S. Pat. No. 6,418,933 B1 discloses an anti-snoring device has maxillary and mandibular bite forms with outwardly extending pivots which are mounted to the bite forms by frameworks which are at least partially embedded in the bite forms.

U.S. Pat. No. 5,499,633 shows two bite forms which may be joined so that the user's mandible projects forwardly of its normal position in order to reduce snoring.

U.S. patent application Publication Number: 2005/0178392 A1 discloses a small piece of cloth tape or other porous hypo allergenic material with a hypo allergenic adhesive on the back is affixed to the lips before sleeping. This may not be effective in preventing the vibration of the soft palate and snoring with or without obstructive sleep apnea.

U.S. Pat. No. 7,016,736 B2 discloses a submental electrical stimulation of the supra hyoid muscles at the floor of the mouth, does not address the snoring due to vibration of the soft palate and uvula.

Numerous management techniques have been described, and none of these treatments have proved adequate. Most of the therapies are inadequate to treat snoring and obstructive sleep apnea. Surgery for the condition is fraught with fear and complications besides high cost and high rate of failure. Hence, the snoring with or without obstructive sleep apnea remains a serious health problem. With increasing obesity (Syndrome X), snoring with or without obstructive sleep apnea is increasing in the general population along with type II diabetes. Accordingly, there has been a need for improved management techniques to reduce or eliminate snoring and obstructive sleep apnea using simple and safe methods. The devices in the present invention are designed to be used to treat snoring and obstructive sleep apnea with minimum or no complications, least disadvantages, and highest compliance.

### SUMMARY OF THE INVENTION

A anti-snoring and anti- obstructive sleep apnea apparatus has a plate or metal frame tongue shelf splint to prevent the flaccid tongue falling back, a palate shelf splint projection to elevate the soft palate and prevents its vibration, incisors teeth receptacles or pockets sockets for jaw displacer to hold the mandible moved forwards, and to prevent it falling back held between the bite block, catheter-tubing to administer oxygen supplementation from the external source which reduces air turbulence and promotes nasal breathing, a submental suprahyoid muscle stimulator placed below the tongue, and above the mucous membrane. An injection port is provided to administer any therapeutic agents and local anesthetics to reduce the sensitivity of the tongue and oral cavity mucus membrane lining to the foreign objects. This device can be used to deliver therapeutic agents to prevent and to treat halitosis and various disease.

It is an object of the present invention is to provide a new, useful, simple, and effective device for the prevention and treatment of snoring and obstructive sleep apnea (OSA).

Another object of the present invention is to provide a safe and effective treatment device which can be self fitted or inserted in the mouth by a snorer and obstructive sleep apnea patients before going to sleep.

A further object of the present invention is to provide a safe and effective treatment device inexpensively to prevent snoring and obstructive sleep apnea.

An important function of this invention is in allowing unobstructed movement of the air through the nose, mouth, and pharynx to the larynx to prevent hypoxia, snoring, and obstructive sleep apnea.

The present invention is to provide an inexpensive method to reduce the incidence of snoring and obstructive sleep apnea that is safe and effective.

The goal of the present invention is to provide an invention that relates to an apparatus inserted into oral cavity which extends all the way to the posterior aspect of the tongue to prevent snoring and obstructive sleep apnea.

The goal of the present invention is where the device is in contact of the tongue and the soft palate and to prevent flaccid tissue closing of the oropharyngeal air way that cause snoring and OSA during sleep.

The device described in this invention comes in contact if the tongue and soft palate becomes flaccid and falls back during sleep to cause snoring and OSA.

The apparatus is placed on the dorsal surface of the tongue in the middle. It can be bent to adjust to the convexity of the tongue upper surface and height of the palate splint-shelf be adjusted for comfort.

The apparatus includes a rounded silicone or balloon or ring on the tip of the apparatus, which comes in contact with the pharyngeal wall if the tongue begins to move backwards during sleep which prevents its movement and snoring with or without obstructive sleep apnea.

The body of the apparatuses is being made as one piece of plastic material, malleable metal, or silicone and other combination synthetic semi synthetic composite material.

The shape of the apparatus conforms to the shape of top part of the tongue, slightly convex upwards to be able to place it easily that it fits snugly.

The convex surface of the apparatus is a convex metal shelf splint or projection (palate shelf splint) splint that prevents the soft palate coming down, and being in touch with the tongue which produces the sound of snoring associated with or without obstructive sleep apnea.

This novel apparatus has a quadrangular, or round metal, plastic plate or metal shelf splint (square ring projection) which is concave in shape so as to fit in the posterior part of the tongue, and prevent the falling off of the tongue back and does not occlude the air passageway drifting inferiorly and posteriorly while sleeping. This tongue shelf splint prevents the passage of air by coming in contact with the pharyngeal wall that produces snoring and obstructive sleep apnea.

It is an object of the present invention is to provide a new, useful, simple, and effective device for the prevention and treatment of snoring and obstructive sleep apnea (OSA).

Another object of the present invention is to provide a safe and effective treatment device which can be self fitted or inserted in the mouth by a snorer and obstructive sleep apnea patients before going to sleep.

A further object of the present invention is to provide a safe and effective treatment device inexpensively to prevent snoring and obstructive sleep apnea device with a lower jaw extending bite block incorporated.

An important function of this invention is in allowing unobstructed movement of the air through the nose, mouth, and pharynx to the larynx to prevent hypoxia, snoring, and obstructive sleep apnea.

The goal of the present invention is to provide an invention that relates to an apparatus inserted into the oral cavity which extends all the way to the posterior aspect of the tongue to prevent snoring and obstructive sleep apnea.

The goal of the present invention where in the device is passed through a bite block. The bite block pulls the lower jaw forwards on fixed upper jaw, hence, prevents the tongue moving downwards and backwards towards the pharyngeal wall so as to cause obstructive sleep apnea.

The device has two “V” angle shaped incisor teeth receivers made of metal or synthetic plastic to fit the upper and lower incisor teeth (incisor teeth receptacles) which can be adjusted to the comfortable levels to pull the lower jaw on the fixed upper jaw to prevent the movement of the tongue backwards which causes snoring and OSA.

It is provided with an oxygen canula to supply oxygen from the oxygen tank or oxygen generator to provide supplementary oxygen at laryngeal inlet in those who are pulmonary function compromised.

It is provided with an injection port and canula to deliver any therapeutic agents and local anesthetics to reduce the sensitivity of the oropharyngeal passages when the device is placed in the mouth before sleeping. The same injection port can be used to deliver therapeutic agents to the surface of the tongue and palate to treat halitosis and other disease afflictions of the oral cavity.

The invention is provided with electrical stimulation to increase the tone of the soft palate and tongue muscles which prevents the flaccidity that takes place during sleep. The novel apparatus includes an electrode to be arranged in the mouth of a patient above the tongue and below the roof of the mouth. The electrode has a novel design. The electrode is coordinated and adapted to the anatomic shape of the surface of the of the tongue running along with the apparatus. In this way, there is surface contact between the electrode and the roof of mouth and surface of the tongue without undesired pressure.

This inventive device is equipped with horse shoe or “U” shaped submental electrical stimulation device to cause electrical muscle stimulating inserted under the tongue to increase their tone so as to prevent them moving backwards which causes obstructive sleep apnea. The additional submental electrical stimulator, arouse the supra hyoid muscles at the floor and root of the mouth which includes electrodes to be arranged below the tongue and above the mucous membrane covering the floor of mouth.

The invention is made up of hypo-allergic, non toxic and non reacting synthetic, natural biodegradable or combinations composite material.

### BRIEF DESCRIPTION OF THE DRAWINGS

The present invention can be well understood with reference to the following drawings. The embodiments of the apparatus or device components in the drawings are not necessarily to scale, stress instead being placed upon visibly and clearly illustrating the principles of the present invention. In the drawings, like reference numerals designate corresponding parts throughout the numerous views of the figures. The purpose of the present invention will become readily valued and implicit from deliberation of the following comprehensive descriptions of the preferred embodiments when taken together with the accompanying drawings, in which:

FIG. 1 is a diagrammatic presentation 100 of the air way during awake and sleeping.

FIG. 2 is a diagrammatic presentation of the air way 200 with soft palate 112 and tongue 114 partially obstructing the airway resulting in snoring.

FIG. 3 is a diagrammatic presentation of the air way 300 with soft palate 112 and tongue 114 completely obstructing the airway resulting in obstructive sleeping apnea (OSA).

FIG. 4 is a view of the diagram 400 showing the device used for stopping snoring and obstructive sleep apnea.

FIG. 5 is a view of the diagram 500 showing the device used for stopping snoring and obstructive sleep apnea with injection port and tongue muscle electrical stimulator.

FIG. 6 is a view of the diagram 600 showing the device in position during sleep to stop snoring and sleep placed in the mouth.

FIG. 7 is the view of the diagram 700 showing the device to stop snoring.

FIG. 8 is the view of the diagram 800 showing the device used for stopping snoring and obstructive sleep apnea with injection port and tongue muscle electrical stimulator.

FIG. 9 is the view of the diagram 900 showing the device in position during sleep to stop snoring and sleep placed in the mouth.

FIG. 10 is the view of the diagraml 000 showing the device to stop snoring and obstructive sleep apnea.

FIG. 11 is the view of the diagram 1100 showing the device used for stopping snoring and obstructive sleep apnea.

FIG. 11a is the view of the diagram 1100a showing the device introduced through a bite block and lower jaw extender.

FIG. 12 is the views of the diagram of the anti snoring and obstructive sleep apnea device expander and insertion forceps or tongs.

### DETAILED DESCRIPTION OF THE INVENTION

According to a present invention snoring and obstructive sleep apnea patients are treated by recognizing a patient with snoring with or without obstructive sleep apnea (OSA) attributable at least in part to due to the vibration of the soft palate during inspiration and movement of a base of the tongue of said patient toward a pharyngeal wall of the patient which causes obstructive sleep apnea. The method includes detecting a region in the tongue extending from mandible to the base of the tongue; preventing the muscular tissue of the tongue moving back towards the pharynx; and vibration of the soft palate resulting in the snoring and obstructive sleep apnea. The present invention prevents the tongue of the patient from obstructing the air passage which causes OSA. The proximal portion of the device is attached to the first to be secured to the teeth by use of Incisors teeth receptacles or pockets of the jaw bone of the patient, or tip of the tongue or hard palate with the rest of the apparatus inserted to along the dorsal surface of the tongue at the same time holding the soft palate and tongue in such position by Palatine and tongue shelf splint that they do not vibrate coming in contact with the tongue or participate in snoring with obstructive sleep apnea.

With reference now to the various figures in which identical embodiments are numbered alike throughout the description of the preferred device of the present invention which will now be presented.

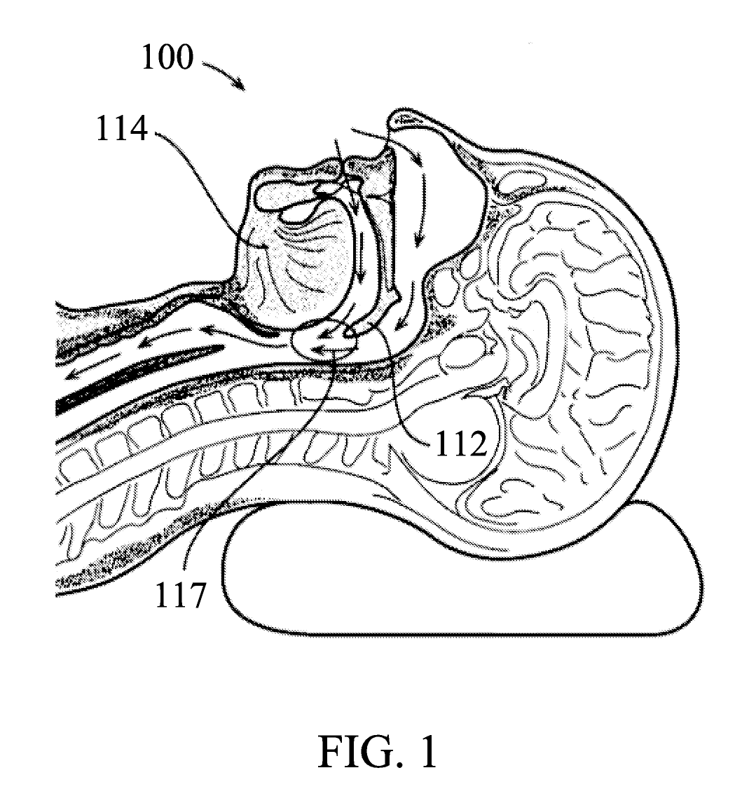

FIG. 1 is the diagrammatic presentation of the normal air way 100 with soft palate 112 and tongue 114 not obstructing the airway passages 117 allowing the free flow of air from the mouth and the nose as the person sleeps on a pillow. The air flow does not produce a physical force like a narrowing of the air stream flow during sleep which does not produce snoring.

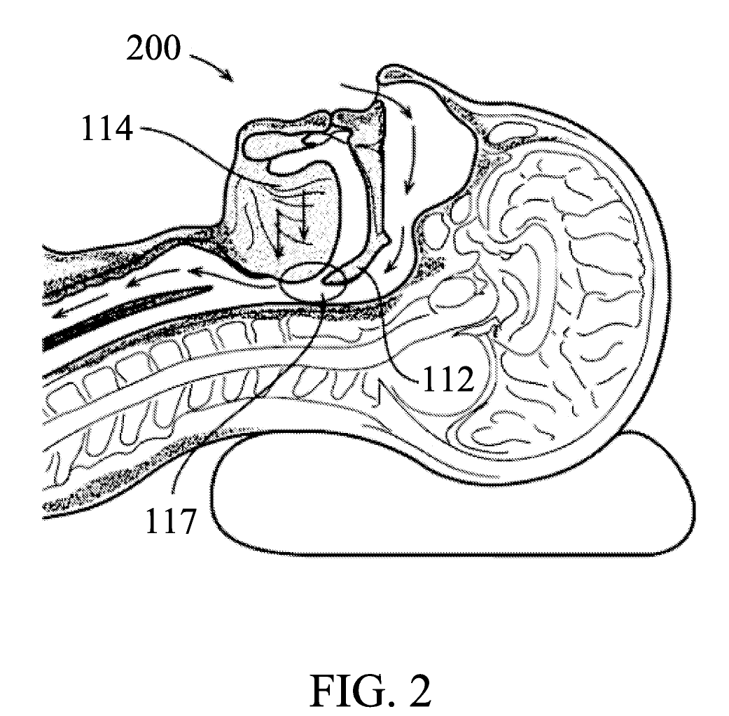

FIG. 2 is the diagrammatic presentation of the air way 200 with soft palate 112 and tongue 114 partially obstructing the airway 117 not allowing the free flow of air from the mouth and the nose as the person sleeps. The air flows in a narrow stream through the air passages 117 vibrating the soft palate 112 and soft tissue around the tongue 114 producing snoring as one sleeps. This is due to relaxation of soft palate 112 and tongue 114 becoming flaccid and falling back to create forcible air stream like Venturi effect which causes vibration soft tissue of the oropharynx, especially the soft palate 112 which does come in contact with narrow stream force to produce sound as one falls asleep.

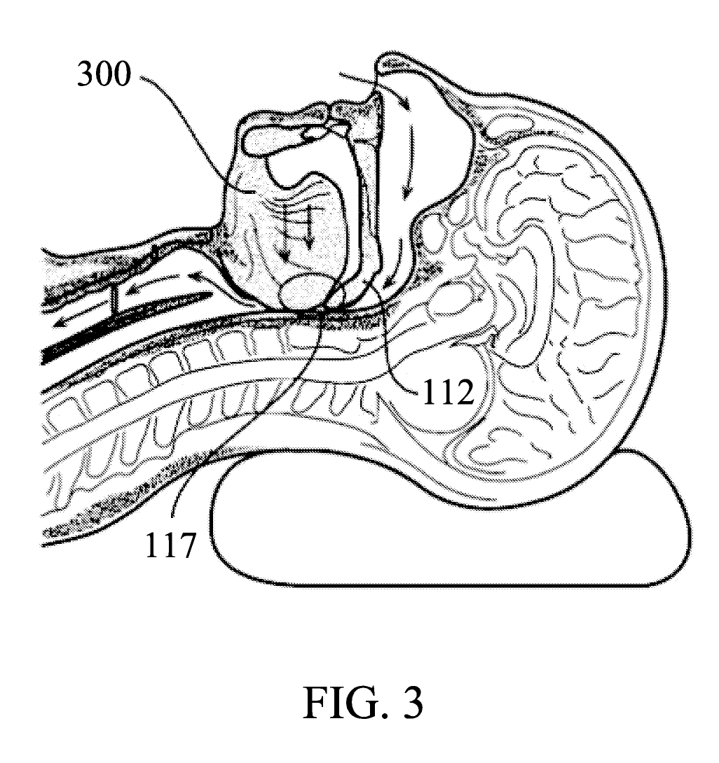

FIG. 3 is the diagrammatic presentation of the air way 300 with soft palate 112, tongue 114 completely obstructing the airway 117 not allowing the free flow of air from the mouth, and the nose to the larynx as the person sleeps. This is due to relaxation of soft palate 112 and tongue 114 becoming flaccid and falling back on the wall of the naso-oro-laryngo-pharynx 117 to create obstruction to passage of air resulting in obstructive sleep apnea (OSA). The person becomes aware, which is due to central nervous system activation, that results in the partial opening of air way allowing the air stream that causes vibration soft tissue of the oropharynx, especially, the soft palate 112 which come in contact with narrow stream force of air that produces snoring sound as one falls asleep. The air flows in a narrow stream or a complete obstruction to air passage 117 which can result in vibrating the soft palate 112 and soft tissue around the tongue 114 producing snoring and complete obstructive sleep apnea.

Our invention as shown in the diagram 400, 500, 600, 700, 800, 900, 1000, and 1100 will prevent the obstruction to the air flow, prevent the palate-uvula vibrating (snoring), and the tongue falling back as described (OSA) in the diagrams by keeping the air way open mechanically which provides the effective method for the treatment of snoring and obstructive sleep apnea.

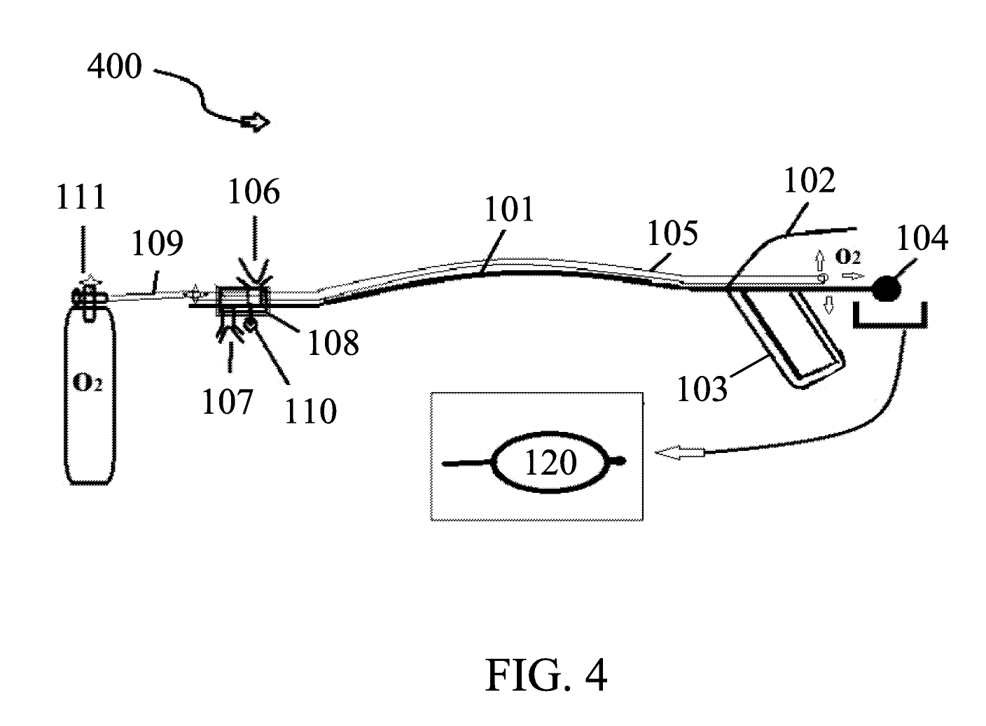

FIG. 4 is the diagrammatic presentation 400 showing the details of the invention for treatment and prevention of snoring, as well as obstructive sleep apnea. It is made of long malleable plastic or metal with flat surfaced long rod or sheet 101 with concavity conforming to the dorsal surface of the tongue. It has a smooth convex metal or plastic rod or thin flat metal plate-projection splint 102 attached to the back third of the device 101 which elevates the soft palate and prevents it from coming in contact with the dorsal surface of the tongue. This prevents vibrating during movement of the air from the nasal or oral passages preventing the snoring. The device is provided with rectangular or round plate or wire frame Tongue shelf splint 103 which is positioned on the posterior inclining surface of the tongue from the back of the tongue to the tongue root. The posterior end of the device 101 has a round metal, plastic, or silicone ball 104 or ring 120 which comes in contact with the pharyngeal wall to prevent the tongue roll back and collapsing of the pharyngeal airway. The metal plate 103 and the ball 104 and ring 120 at the end of the device will prevent the movement of the tongue falling back and collapsing pharyngeal airway and thus prevents OSA. Soft palate shelf splint attachment 102, backward tongue movement preventer tongue shelf splint 103 with metal, plastic ball, or a balloon 104 or ring 120 abutting against the pharyngeal wall prevents the obstruction to the airway (OSA) and prevents the vibration sound by the movement of the soft palate. Instead of a round silicone ball, this extension from device 101 can be provided with oval ring 120 with a small silicone ball at 3 O'clock positions. This ring shaped attachment at the end will prevent the collapse of the soft tissue of oropharynx on the airway and prevent OSA development. The device 101 is provided with an oxygen supply tubing 105 connected to oxygen tank 111 or air oxygen concentrator by delivery tubing 109. The device 101 is provided with two adjustable incisors teeth receptacles or incisors teeth sockets for upper 106 and lower jaw incisors 107. They prevent the lower jaw from drifting inferiorly and posteriorly during sleep. They can be moved to adjust the distance between the upper and lower jaw to draw the lower jaw forward which prevents the falling of the flaccid tongue backwards during sleep. The teeth socket 108 allows the incisor teeth can be moved to adjust the distance for convenience. When moved, it is provided with screws 110 to hold it in place when in use. The palate shelf splint 102 and the tongue holder shelf splint or tongue blocker 103 can be provided with electrical wires which pass milliamps of electricity to maintain the tone of the soft palate and tongue muscles.

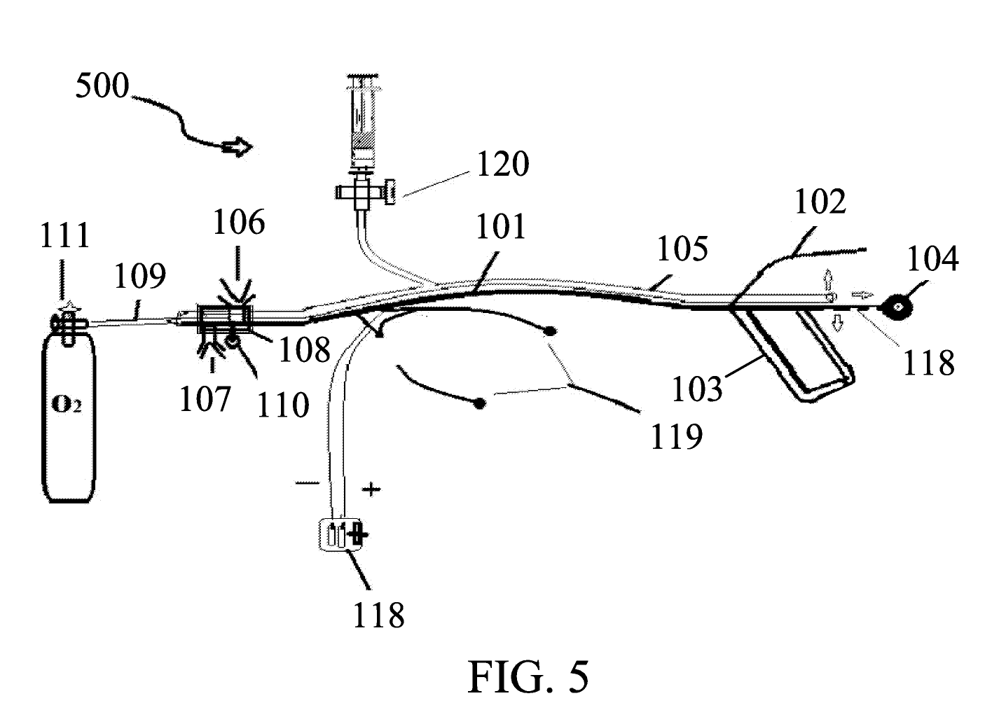

FIG. 5 is the diagrammatic presentation 500 showing the details of the invention for treatment and prevention of snoring with or without obstructive sleep apnea. The explanation is the same as FIG. 4, except, this device is equipped with horse shoe or “U” shaped submental electrical stimulation device 119 to cause electrical muscle stimulating inserted under the tongue to increase their tone and to prevent them moving backwards which causes obstructive sleep apnea. This attachment is for submental electrical stimulation of supra hyoid muscles at the floor of mouth. It includes an electrode to be arranged below the tongue and above the mucous membrane covering the floor of mouth below the anterior one third of the tongue and in front of the root of the tongue. The electrode includes a form body being made as one piece of plastic material with metal conductor inside the plastic material. The surface portion is designed to be electrically conductive. It is designed to form the anatomic shape of the floor of the mouth in the region of the lower jaw curve around the frenulum of the tongue on the sides of the tongue encircling the root of the tongue. The surface of this addition to the device is electrically conductible. The surface portion has the shape of a “U” or Horseshoe with two free arms surround the frenulum of tongue on both sides. The electrical stimulation through these arms of the device increase the tone of these tongue muscles especially suprahyoid muscles and prevents the tongue falling back and obstructs the airway resulting in snoring and obstructive sleep apnea. It is connected to electricity control device boxl 18 which has ON and OFF switch with milliamps (mA) of electrical current delivery adjuster. This will help to deliver the comfortable and tolerable levels of electricity without disturbing the sleep. This horse shoe shaped attachment with electrical connections is made of long malleable plastic or metal components 119. It has concavity inside to fit the root of the tongue without discomfort.

This device 500 in the FIG. 5 is provided with infusion canula attached to the syringe 120 with a stop cock to inject any therapeutic agents and local anesthetic to anesthetize the sensitive posterior part of the tongue, oropharynx, and epiglottis, to deliver therapeutic agents to treat halitosis and to treat disease afflictions of the tongue and palate. The device 101 is provided with oxygen supply tubing 105 connected to oxygen tank 111 or air oxygen concentrator by delivery tubing 109. The main body of the apparatus is hollow and is provided with perforations 121 to allow the supplemental oxygen 111 delivered close to the laryngeal air inlet.

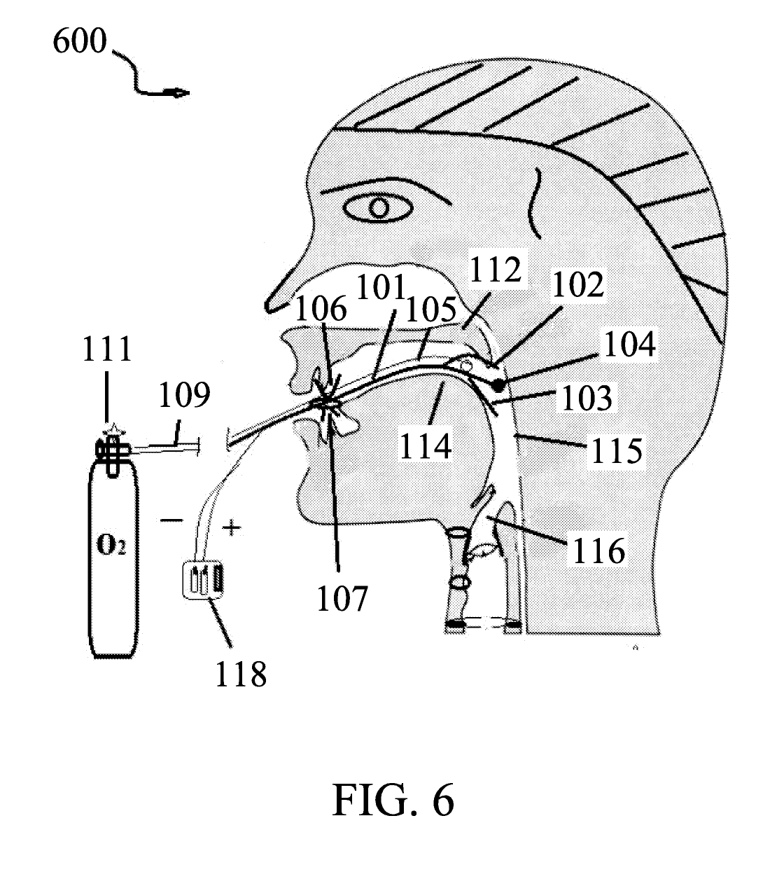

FIG. 6 is the diagrammatic presentation 600 showing the details of the invention for treatment and/or prevention of snoring and obstructive sleep apnea positioned inside the oral cavity (mouth) during sleep. It is made of long malleable plastic or metal rod or flat sheet 101 with concavity conforming to the surface of the tongue 114. It has smooth convex palate metal or plastic shelf splint 102 attached to the distal part of the device 101 which elevates the soft palate and uvula 112 and prevents it from vibrating during movement of the air from the nasal, oral, and laryngeal passages thus preventing the snoring. It is provided with rectangular or round plate or wire tongue shelf splint 103 which is positioned on the posterior inclining surface of the tongue. The end of the device 101 has a round metal or plastic or silicone ball 104 (or oval frame in FIG. 4, 120) which comes in contact with the pharyngeal wall 115 if the tongue becomes flaccid during sleep and attempt to roll back to create obstruction to the air passages 116 through the pharynx 115 to the laryngeal opening 116. The metal plate 103 and the ball at the end prevents the movement of the tongue falling back. Soft palate shelf splint 102, backward tongue shelf splint movement preventer 103 with metal, plastic ball, or a balloon 104 at the end of the device abutting against the pharyngeal wall 115 prevents the obstruction to the airway. This prevents the vibration sound by the movement of the soft palate and obstructive sleep apnea. The device 101 is provided with oxygen supply tubing 105 connected to oxygen tank 111 or air oxygen concentrator-generator by delivery tubing 109. The device 101 is provided with two Incisors teeth receptacles or incisors teeth sockets for upper 106 and lower jaws 107 incisor teeth. They can be moved to adjust the distance between the upper and lower jaw to draw the lower jaw (mandible) forwards, which prevents the falling of the flaccid tongue backwards during sleep. The teeth socket 108 shown in FIG. 1 can be moved to adjust the distance for convenience. When moved, it is provided with screws 110 to hold it in place. The palate shelf splint 102 and the tongue shelf splint 103 can be provided with electrical wires which pass milliamps of electricity to maintain and to increase the tone of the palate and tongue muscles. The present invention has milliamps of electricity generator from the battery pack 118 with ON and OFF switch to transmit electricity (+, −) to palate 112 through 102 palate shelf splint and tongue shelf splint or restrainer 103 to maintaining the tone of these muscle and to prevent them falling back on the pharynx and to prevent snoring with or without obstructive sleep apnea.

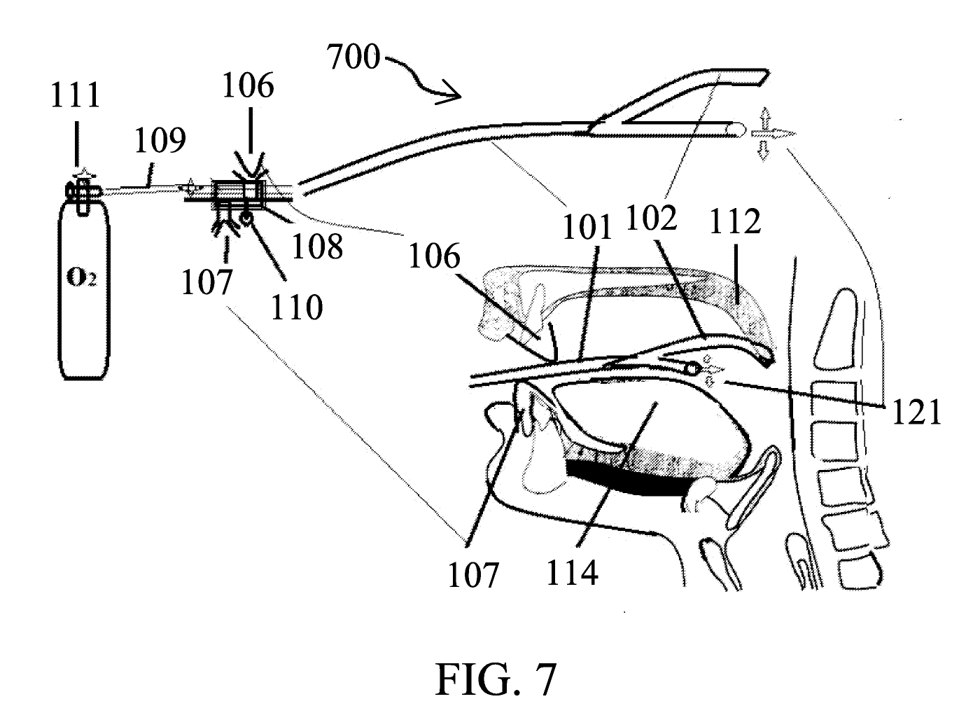

FIG. 7 is the diagrammatic presentation 700 showing the details of the invention for treatment or prevention of snoring with or without obstructive sleep apnea positioned inside the mouth during sleep. This is a simplified version that is explained in FIGS. 4, 5, and 6. This is suitable as anti snoring device. When it incorporates jaw moving attachments like in the diagrams 106, 107, 108, into the design, it can move the lower jaw forwards which can prevent the tongue moving backwards during sleep and acts anti obstructive sleep apnea device. Without the use of the attachment 108, it is an effective simple anti snoring devise. Note: the flat curved palatine shelf splint attachment 102 lifts the uvula and soft palate 112, thus, effectively controlling vibration of the palate during inspiration and prevents the snoring. It has centrally placed canula in the device 101 to deliver the oxygen from the oxygen tank 111 and connecting tubing which is delivered at the back end of the device 121 as shown in the diagrams. It will supply the supplemental oxygen in pulmonary compromised patients and prevent any anoxic-hypoxic ill effects during sleep.

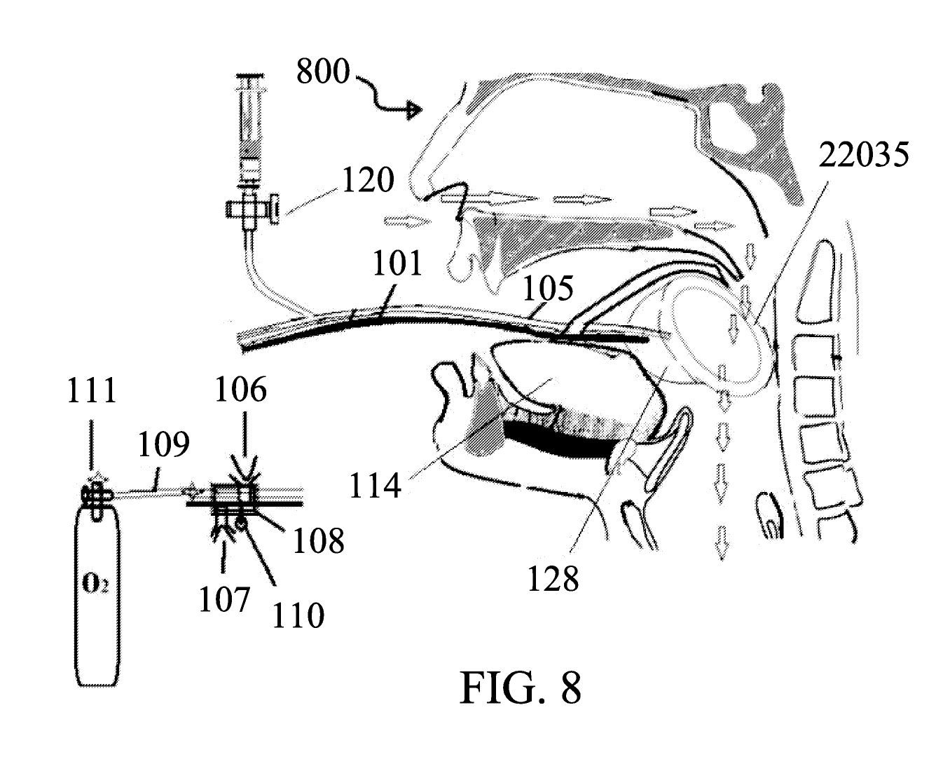

FIG. 8 is the diagrammatic presentation 800 showing the details of the invention for treatment or prevention of snoring with or without obstructive sleep apnea positioned inside the mouth during sleep. This is similar device shown in FIG. 7, except, it has ring shaped balloon 22035 with a central opening to allow the air from the nose to the larynx. When inflated, the balloon abuts against the oropharyngeal wall and the balloon in front of it is the tongue balloon 128 which prevents the tongue moving backwards which prevents snoring and sleep apnea. It is provided with inflating canula and supplementary oxygen canula to be provided as needed. It is provided with injection port 120 to deliver therapeutic agents and anti halitosis agents.

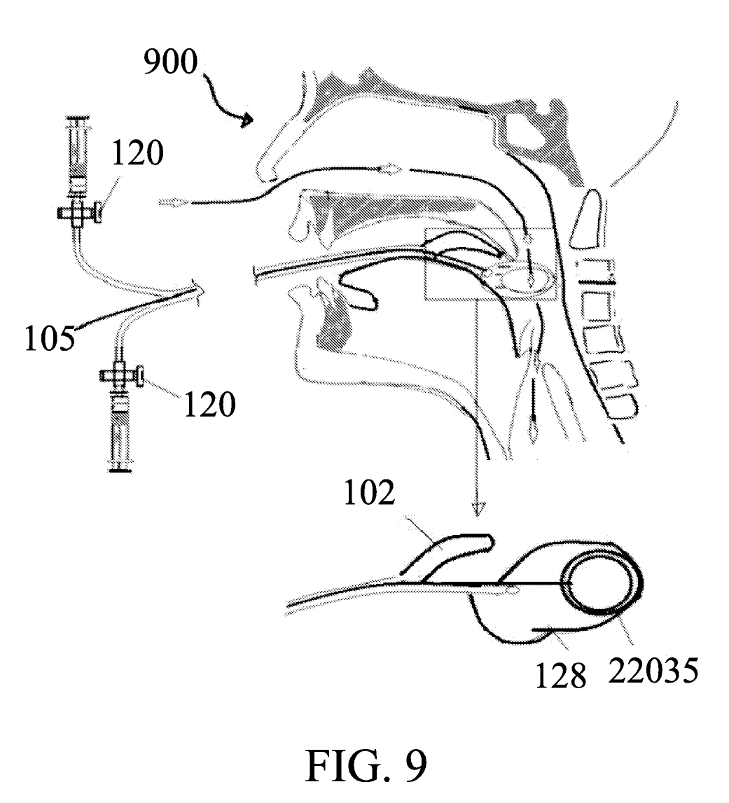

FIG. 9 is the diagrammatic presentation 900 showing the details of the invention for treatment or prevention of snoring with or without obstructive sleep apnea positioned inside the mouth during sleep. It is similar to the device explained in the FIG. 9, except, it has metal or rigid plastic ring to have a non collapsible metal ring 22035 with tongue balloon attached to the proximal part of the pharyngeal metal or rigid plastic ring. Arrows points to the direction of the air entering from the nose to the larynx through the ring 22035 which prevents the soft tissue obstruction in the oro-Nasopharynx for the passage of the air which is responsible for obstructive sleep apnea due to loss of tone of the muscles during sleep. It provides supplemental oxygen 120 for pulmonary function compromised patients.

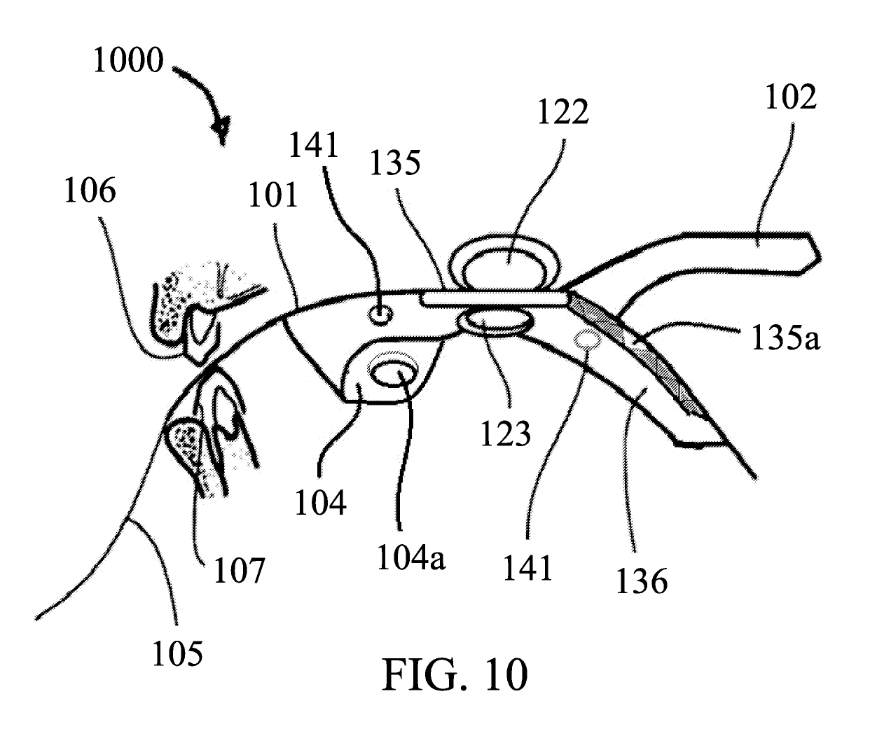

FIG. 10 is the diagrammatic presentation 1000 showing the details of the invention for treatment of snoring, and obstructive sleep apnea. The device 101 has a cap or hood 104 which covers the free margin of the tip of the tongue like a cap and short skirt 136 which covers the sides and free margins of the tongue all the way to its root. The cap 104 can be provided with suction cup 104a which can attach the device to the undersurface of the tip of the tongue and hold it without sliding or slipping. The free margins of the tongue tip cap 104 may be provided with an elastic band to encircle the free floating part of the tongue which will hold the device in close contact like an elastic ball has a flat shelf splint 102 which holds the soft palate and uvula lifted up to prevent the sound production (snoring) during sleep. It has dorsal 122 and ventral 123 suction cups attached to the plastic plate 135 which attaches the device to the hard palate and dorsal surface of the tongue. This plastic plate can be extended back from the posterior end as tongue shelf of split 135a which can also prevent the backward movement of the tongue and prevent OSA. It can be made up of metal or malleable synthetic material so as to bend it to fit the back part of the tongue snugly and prevent it sliding back while asleep. In such a case the displacement of the mandible by 106 and 107 teeth pocket forwards may not be needed if this extension 135a is used. This 135a may be attached to the plastic plates 135 with a tight hinge to facilitate its movement over fixed plastic plate to allow the fitting of the device on the back part of the tongue. The device is provided with upper 106 and lowers 107 incisor teeth sockets pocket which will facilitate the holding of the lower jaw (mandible) moving forwards over the fixed upper jaw. It has a string 105 attached at the front end to prevent it being accidentally swallowed or aspirated during sleep. The device has 4 holes 141 in the skirt 136 for inserting of the tongs tips or forceps for opening the device and placing it on the dorsal aspect, the tip and the sides of the tongue with ease before going to sleep.

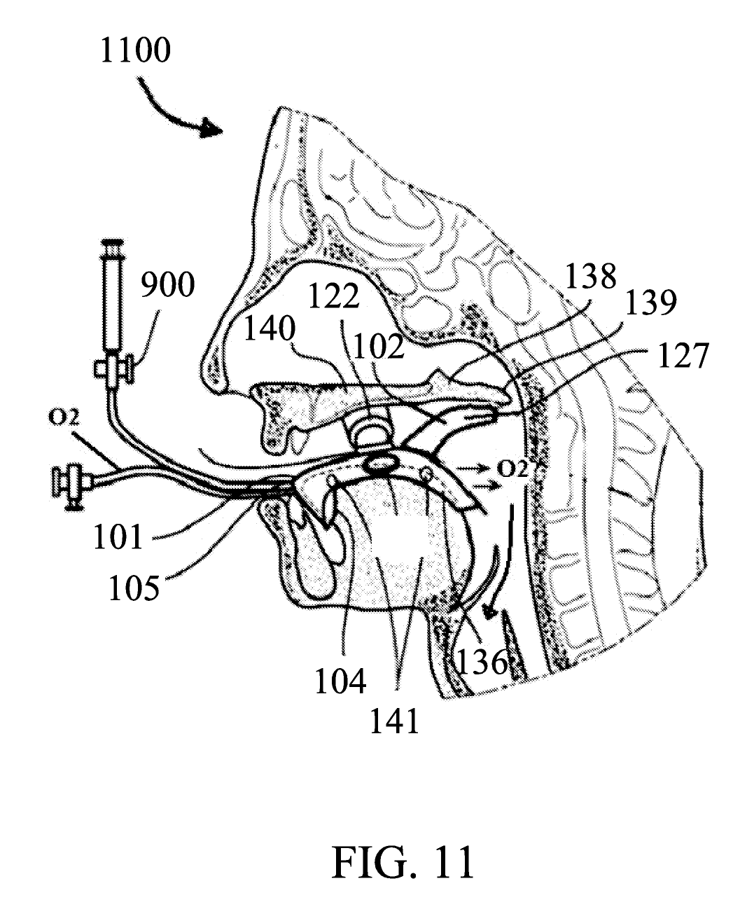

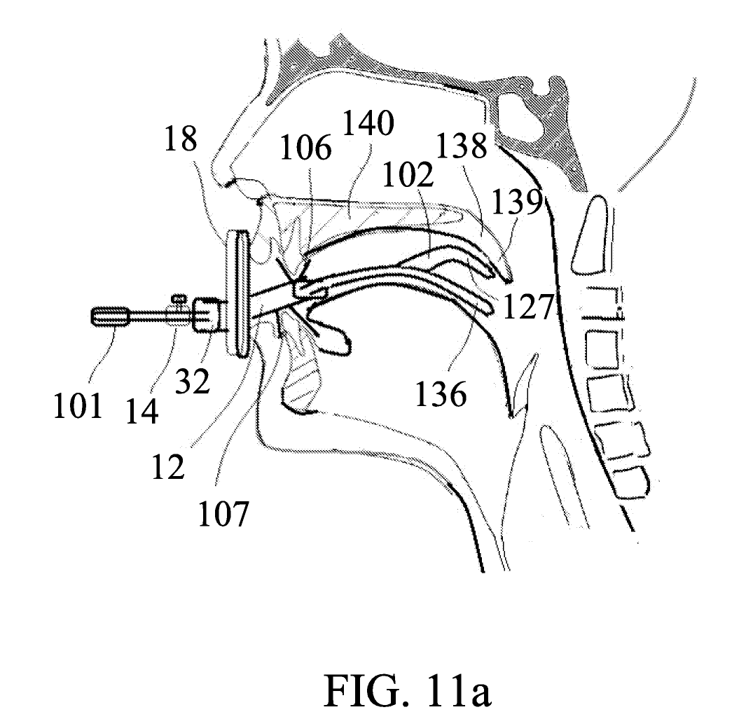

FIG. 11 is the diagrammatic presentation 1100 showing the details of the invention for treatment of snoring, and obstructive sleep apnea device from FIG. 10 placed in the oral cavity in operation. It is another type of anti snoring and anti obstructive sleep apnea device 101 which has only shelf splint on the back of the device which can be adjusted up or down to the comfort of the user. The device does have suction vacuum cups. The device is placed on the tongue and the shelf splint 102 lifts the soft palate 138 and the projection 127 pushes up the uvula 139 and prevents it coming in contact with the back of the tongue, and prevents their vibration that produces snoring. The suction cups 122 and 123 along with the cap 104 attached the device to the hard palate 140 and tongue which prevents the tongue moving backwards and downwards when it becomes flaccid during sleep and holds it in its root. Thus, the device in place prevents the tongue moving inferiorly and posteriorly towards the oropharynx during its flaccid state for the duration of the sleep and prevents the obstructive sleep apnea and snoring. Use of this device may obviate the use of incisor teeth sockets to move the lower jaw forwards. The tongue hood or cap 104 holds the device in position without displacement or sliding backwards during sleep. It provides with string 105 attached to the device to prevent accidental swallowing or aspiration during sleep. The device has 4 holes 141 on the skirt 136 for inserting of the tongs tips or forceps for opening the device and placing it on the tongue with ease before going to sleep. It has injection canula 120 to deliver any therapeutic agents on the surface of the tongue or local anesthetics to reduce the sensitivity of the tongue to the device. The oxygen delivery canula to administer the supplemental oxygen if need be.

FIG. 11a is the diagrammatic presentation 1100a showing the details of the invention for treatment of snoring, and obstructive sleep apnea device with a jaw extending bite block 12. The explanation is same for this as described in the FIGS. 4, 5, 6, and 7. In addition, this anti snoring and obstructive sleep apnea device includes additional embodiment. It includes a bite block 12, with incisor teeth receptacles situated on the upper 106 and lower 107 surface of the bite block. The distance of the lower 107 incisors teeth receptacles is movable to adjust the forward movement of the lower jaw to pull the tongue forwards to prevent it sliding back to the pharyngeal wall to cause snoring and obstructive sleep apnea. It has planer surface 18 attached to the proximal end of the bite block comes in contact with the upper and lower lips, which prevents bite block moving inside the oral cavity. The planer surface is the front surface of the bite block 12. The planer surface has a hole 32 with extends from the proximal 32 to the distal end of the bite block 12 which allows the easy movement of the device to adjust the distance of the palate and tongue deflector from the lip to the soft palate 102, 127, 138, and uvula 139, and back of the tongue 136. Once the desired distance is reached, there is a distance adjusting screw 14 which holds the device 101 in position. This hole 32 in the bite block 12 also accommodates oxygen and therapeutic agent's delivery canula if the person who uses this device is on supplemental oxygen and to treat diseases of the tongue and oral cavity. The sides of the planer surface 18 has two perforations (holes) located at 3 and 9 O'clock position at the center of the edge, which will allow to bind the device to the headband to keep the device in position with elastic band and velcro during sleep.

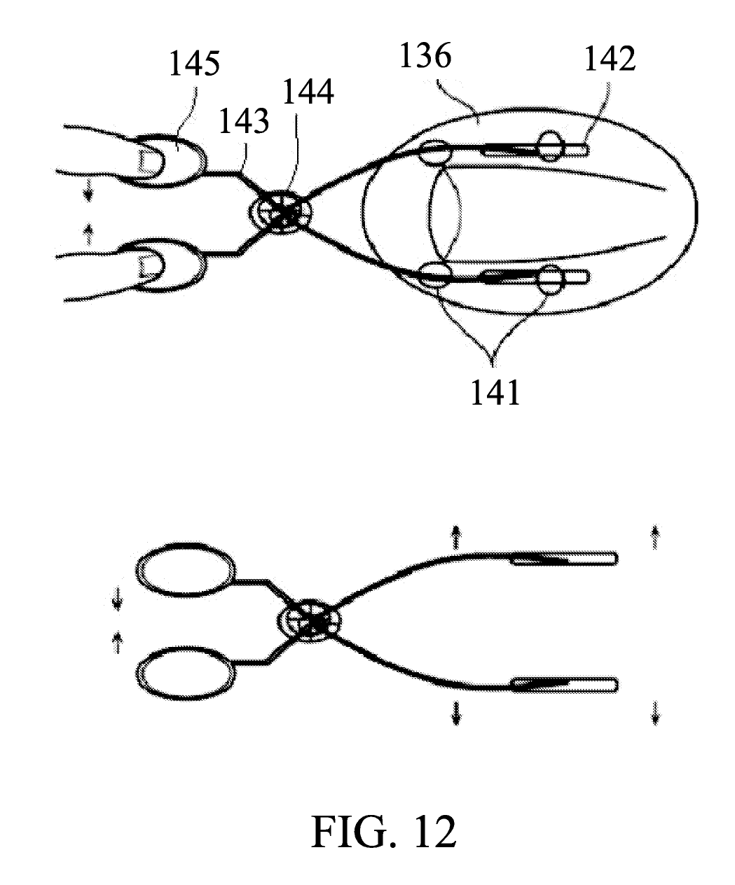

FIG. 12 is the diagram of the anti snoring and OSA device insertion-placement forceps (tongs) 1200 showing the approximating finger holes in the handle, the tip open up (arrows) and expands the device. The snoring and obstructive sleep apnea placement forceps have 4 components described as follows:

1) Blades: The blades 142 are flat which hold the device passing though the holes 141 to grasp the device. Each blade has curvature to fit around the tongues outside convex curvature.

2) Shanks: The shanks 143 connect the blades to the handles and to provide the length of the device to enter the oral cavity. They are parallel, crossing at the lock.

3) Lock: The lock 144 is the articulation between the shanks with tightening screws to hold the blades containing anti snoring and obstructive sleep apnea device in position.

4) Handles: The handles have two finger holes 145 where the operator holds the device opened with thumb and index finger, tighten the lock, holds it on the tip of the tongue, and inserts the device on the tongue gently. While using this device placement forceps; the blades tips are passed through the four side holes 141 in front and back of the side of the device sleeve/skirt 136. Then the forceps tips are expanded to open the device to be positioned on the tongue from tip, side and all the way to the back of the tongue as visualized in the FIGS. 11 and 12 with ease. After each use, wash the device placement forceps' with warm water and soap or place in platter and immerse in oral antiseptic mouth wash solution. Wash it before using in tepid water.

Our invention as shown in the diagrams 400, 500, 600, 700, 800, 900, 1000, 1100 and 1100a will prevent the obstruction to the air flow to the air passages; prevent the palate vibrating responsible for snoring and tongue falling back which contributes to both snoring and OSA as described in the diagrams 2, and 3 by keeping the nasal-oral-laryngeal air way open mechanically. These inventive devices act as anti snoring and anti obstructive sleep apnea device.

The undersurface of the device specially the posterior one third of the device can be coated with antibacterial, hypo allergic and antiviral antiseptic therapeutic agents to prevent the growth of the bacteria and other infecting agents and to prevent the production of halitosis. The various therapeutic agents for specific treatments of the tongue and oral cavity afflictions can be applied to the undersurface of the device as part of the treatment modality. They can deliver therapeutic agents and oxygen to the undersurface of the tongue from outside once the device is on the tongue through the canula provided with three way stopcock 120.

### PREPARATION OF THE PATIENT AND EMPLOYING THE DEVICS

Examine the patient thoroughly, especially, oropharyngeal area, nose, and throat for any medical and mechanical conditions that can predispose to snoring with or without obstructive sleep apnea. If there is a contributing factor found, and correctable, the patient should be advised to seek medical help to correct the issues when using the devices for treatment of snoring and obstructive sleep apnea. If the patient has undergone sleep studies to diagnose sleep apnea, the report needs to be obtained to gauge the severity of the OSA. The patients should be advised to stop smoking, to avoid the use of narcotics, sleeping medications, hypnotics, sedative, sleep causing antihistamines, and alcohol before going to bed. To avoid food regurgitation, which can add to the pathophysiology of OSA, the patient must eat a moderate dinner about 3-4 hours before going to bed. A patient should avoid watching the TV and sleep in a quiet room without any external disturbances that can affect your sleep. The patient should use stomach acid production blockers to prevent the GERD and to prevent regurgitation of the stomach contents which can cause air way problems including aspiration pneumonitis.

The following advice is given before use of anti snoring and obstructive sleep apnea devices such as 1. Brush the teeth, 2. Use floss to clear the gums. 3. Use tongue scraper to clean the tongue of any coating. 4. Use a mild mouth antiseptic wash.

Soak the device in an antiseptic and coat it with lubricant if needed, (oily coating which is not toxic such as olive oil). If the device is not tolerable, due to sensitivity, patient may have to use local anesthetic lozenges, which are available over the counter. If still difficult to use the device due to sensitivity of the oropharyngeal passages, get local anesthetic jelly or spray (Citanest spray) through your physician. Wait till the local anesthetic takes effect and then position it in the mouth. Avoid eating food if local anesthetic spray is used till it wears off.

Use a well lighted mirror to position the device to place the soft palate elevator shelf splint is positioned appropriately at correct position in contact with the oral undersurface of the soft palate and uvula. It will facilitate how deep you need to pass device 101 as it is passed on the tongue and back of the tongue. The tip of the device used for snoring with or without obstructive sleep apnea needs to touch the posterior wall of the pharynx in some of the embodiments. It is a safety mechanism which is pushed back to hold the tongue forwards and prevent it move any further will cause OSA. Make sure the front end is tied to a string provided that it won't be accidentally swallowed while sleeping. Once, you get used to the use of the device for a week or two, you may not need any more topical numbing medications. Please use only when needed. With habitual use of the device repeatedly will reduce or eliminate the gag reflex completely with no need to use local anesthetics. Keep the device immersed in antiseptic mouthwash and wash in clean warm water before placement. If oxygen supplementation is needed in pulmonary compromised patients, use an oxygen concentrator or 100% oxygen from the cylinder and keep the flow to the minimum required levels that it does not disturb sleep.

Insert the device, to prevent snoring and the patient needs to insert it deep into the throat to prevent snoring associated OSA. The distance can be adjusted according to the tolerability, avoiding gag reflex and convinces. For snoring with obstructive sleep apnea, the tongue shelf splint restrainer or blocker 103 need to be carefully positioned properly to prevent the tendency of the flaccid tongue falling or moving posteriorly and inferiorly to cause obstruction to the upper respiratory passages which causes snoring and obstructive sleep apnea. Only way to prevent tongue falling back from the floor of the mouth to prevent obstructive sleep apnea is by mechanical obstruction to prevent the flaccid tongue move backwards during sleep (especially in supine position) provided by our inventive device.

If there is no OSA, use the device without the, back tongue shelf splint to prevent the movement of the tongue 103 with metal, plastic ball, or a balloon 104 abutting against the pharyngeal wall need not be used. If the device does not work, use the device which incorporates backward tongue movement preventer shelf splint 103. The soft palate becomes flaccid and moves downwards coming in contact with the back of the tongue and oropharynx when the patient sleeps to produce snoring which is prevented by use of this anti snoring invention.

The anti snoring and obstructive sleep apnea device may be made of thermoplastic or elastomeric resin or synthetic plastic or silicone resins with or without metal component such as stainless steel, aluminum, and copper rods added to strengthen the composite materials and add moldability to the user's mouth. It should not be too rigid, must be soft, may be made up of silicone, it must be fairly malleable to prevent trauma to the tongue and palate with night long use.

Use forceps as described in FIG. 12 to facilitate the placement of the device as seen in the FIG. 10 on the surface of the tongue appropriately. The forceps blades are passed through the holes on the skirt 141, expand the device, and insert it to the protruding tongue as shown in the diagram 11. The insertion of the device is done easily in front of a mirror for the convenience.

Keep the forceps cleaned and store in a dry place or in antiseptic or mouth wash bowl.

### ADVANTAGES OF THE CURRENT ANTI SNORING AND ANTI OBSTRUCTIVE SLEEP APNEA INVENTION

Advantage of the present invention is that it is available for an anti-snoring and OSA therapies having an external and internal oral device to reduce or eliminate snoring with or without obstructive sleep apnea during sleep.

An added benefit of the present invention is that it provides for an anti-snoring and OSA device that is easily self-adjustable and does away with the need for professional and laboratory assistance or clinician fabrication.

Another benefit of using this invention is that it has provision for supplemental oxygen for those who have severe pulmonary diseases needing high concentration of supplemental oxygen to prevent any adverse health effects due to OSA hypoxia during sleep.

Yet another advantage of the present invention is that it provides for an anti snoring and OSA combined device that is fabricated from a thermoplastic material (elastomeric resin) with or without metal component, which can be easily shaped to fit the anatomy of the oral cavity, tongue, and the soft palate.

An extra benefit of the present invention is that it provides for an anti snoring and OSA combined device which is moldable after immersion in boiling water so that it can be adapted by the user to have a comfortable and individualized fit.

An additional benefit of the present invention is that it provides for an anti-snoring and OSA device that can be coated with therapeutic agents that prevents and treats bad breath and can be used to treat the disease afflictions of the tongue and palate. The therapeutic agents can be delivered through the three way stopcock and syringe provided in the device after placement of the device before going to sleep.

An additional benefit of the present invention is that it provides for an anti-snoring and OSA device that has an intra oral dental overlay structure-incisors teeth receptacles or pockets which can be used displaces the lower jaw to the comfortable level at the same time supports the tongue against the user's palate to keep the palate from reverberating during mouth breathing to prevent snoring and OSA by the user.

A further advantage of the present invention is that it provides for an anti snoring and OSA device which can be easily used, stored, cleaned, and mass produced economically to make it affordable for millions who suffer from snoring with or without obstructive sleep apnea.

Numerous modifications; alternative arrangements of steps explained and examples given herein may be devised by those skilled in the art without departing from the spirit and the scope of the present invention. The appended claims are intended to cover such modifications and arrangements. Thus, the present invention has been described above with particularity and detail in connection. This is presently deemed to be the most practical and preferred embodiments of the invention. The invention will be apparent to those of ordinary skill in the art that numerous modifications, including, but not limited to, variations in size, materials, shape, form function, and manner of procedure, assembly, and the use may be made. The preferred embodiment of the present invention has been described. The invention should be understood that various changes, adaptations, and modifications may be made thereto. It should be understood, therefore, that the invention is not limited to details of the illustrated invention. This method can be used to treat snoring with or without obstructive sleep apnea and prevent the health hazards associated with the conditions. The preferred embodiments of the present invention have been described. This should be understood, therefore, that the invention is not limited to details of the illustrated invention examples. Other features and advantages of the present invention will become apparent to one with skill in the art upon examination of the descriptions and drawings. It is intended that all such additional features and advantages be included herein within the scope of the present invention, as defined by the claims.

What is claimed is:

1. An anti-snoring and anti-obstructive sleep apnea device comprising:
a. an oral device portion adapted to removably cover the top portion of the tongue;
b. a moldable oral device for maintaining the tongue away from the soft palate and oral pharynx to prevent air flow from causing the palate to reverberate during breathing to cause snoring and to prevent the tongue from moving back which causes obstructive sleep apnea;
c. a plate shelf splint portion centrally disposed on said oral device portion;
d. a malleable ring attached to the distal end of the hard palate plate which prevents the movement of the tongue backwards acting as a splint; whereby snoring and obstructive sleep apnea are prevented during use.

2. The anti-snoring and anti-obstructive sleep apnea device according to claim 1 further comprising: a first suction cup disposed on upper surface of said plate portion wherein said first suction cup removably holds said oral device portion to a dorsal surface of said tongue tethered to the hard palate preventing the sliding of the tongue backwards.

3. The anti-snoring and anti-obstructive sleep apnea device according to claim 1 further comprising: a second suction cup disposed on a lower surface of said plate portion wherein said second suction cup removably holds said oral device portion to a dorsal surface of said tongue tethered to the hard palate, thus, preventing the sliding of the tongue backwards.

4. The anti-snoring and anti-obstructive sleep apnea device according to claim 1 further comprising an extension having a round end adapted to reach the pharyngeal wall and prevent the tongue moving back.

5. The anti-snoring and anti-obstructive sleep apnea device according to claim 1 wherein said first and second suction cups are adapted to hold a dental adhesive.

6. The anti-snoring and anti-obstructive sleep apnea device according to claim 1 further comprising:
a. an inflatable balloon disposed on a posterior portion of said plastic plate;
b. said inflatable balloon being adapted to contact a portion of a back portion of said tongue wherein said tongue is prevented from moving backwards whereby snoring and obstructive sleep apnea are prevented during use; and
c. said inflatable balloon having an airway opening centrally disposed, wherein, air is allowed to move through said airway opening from a nose to a larynx.

7. An anti snoring and anti obstructive sleep apnea device in accordance with claim 1, further comprising a cannula with three way stopcock adapted to inflate said inflatable balloon with a fluid.

8. An anti snoring and anti obstructive sleep apnea device in accordance with claim 1 further comprising an injection cannula with three way stopcock adapted to inflate said balloon and to deliver at least one of antibiotics, antiseptics, local anesthetic and therapeutic agents on to the surface of the tongue to treat any oral pathology and prevent gag reflex.

9. An anti-snoring device in accordance with claim 1, further comprising an obstructive sleep apnea tongue shelf splint extension attached to a body portion disposed at a junction of a posterior one third and anterior two thirds, which prevents the tongue moving backwards to prevent occlusive sleep apnea due to flaccid tongue falling.

10. An anti-snoring device in accordance with claim 1, further comprising an extension with a round structure at the end therein adapted to come in contact with an oropharyngeal wall and prevents the further movement of the device with said tongue.

11. The anti-snoring and anti-obstructive sleep apnea device according to claim 1 further comprising:
a. a hood portion adapted to fit across a tip of said tongue wherein said oral device portion is prevented from moving back on said tongue;
b. said hood portion being disposed on a forward portion of said oral device portion to cover said tip of said tongue and hold said device in place;
c. a skirt portion which covers the sides of said tongue;
d. said skirt having two holes on each side to accommodate the insertion of forceps on said tongue without difficulty; and
e. a thread attached to said tip to prevent dislocation therein.

12. The anti-snoring and anti-obstructive sleep apnea device according to claim 1 further comprising a safety portion attached to said oral device portion wherein said anti-snoring and anti-obstructive sleep apnea device is prevented from being accidentally swallowed or inhaled during sleep.

13. The anti-snoring and anti-obstructive sleep apnea device according to claim 1 further comprising:
a. a plurality of oxygen delivery exits disposed on a posterior portion of said oral device portion; and
b. oxygen delivery tubing portion in communication with said oxygen delivery exits and oxygen source, wherein, hypoxia is prevented especially in pulmonary compromised patients.

14. The anti-snoring and anti-obstructive sleep apnea device according to claim 1 further comprising;
a. a tooth socket;
b. said tooth socket having a “V” -shaped configuration, whereby an incisor tooth fits within said tooth socket wherein said tongue is prevented from sliding back.

15. The anti-snoring and anti-obstructive sleep apnea device according to claim 1 further comprising a metallic strip embedded within said oral device portion wherein said oral device portion is strengthened.

16. The anti-snoring and anti-obstructive sleep apnea device according to claim 1 wherein said oral device portion is made of moldable thermoplastic.

17. An anti snoring and anti obstructive sleep apnea device in accordance with claim 1 further comprising:
a. a bite block adapted to fit between incisor teeth, and
b. said bite block having incisor teeth sockets located on said bite block wherein a lower jaw is held in a selected position with respect to an upper jaw.
